(19) United States
(12) Patent Application Publication
(10) Pub. No.: US 2019/0388242 A1
(43) Pub. Date: Dec. 26, 2019

(54) Interbody Inserter

(71) Applicant: Nexus Spine, L.L.C., Salt Lake City, UT (US)

(72) Inventors: Jeff Harris, Salt Lake City, UT (US); Peter Halverson, Draper, UT (US); David Hawkes, Pleasant Grove, UT (US)

(21) Appl. No.: 16/452,512

(22) Filed: Jun. 25, 2019

Related U.S. Application Data
(60) Provisional application No. 62/689,707, filed on Jun. 25, 2018.

(51) Int. Cl. A61F 2/46; A61F 2/44

(52) U.S. Cl. CPC A61F 2/4611; A61F 2/447; A61F 2002/30143; A61F 2002/4623; A61F 2002/4627; A61F 2002/4475

(57) ABSTRACT

An implant inserter is adapted to secure and facilitate insertion of a surgical implant. The implant has an inserter attachment interface having a narrow external opening at a surface thereof and a broader internal opening. The implant inserter includes a handle and an inserter shaft. The implant inserter also includes a pair of flexible tabs extending from a distal end of the inserter shaft with laterally extending protrusions adapted to extend into the broader internal opening and an expansion shaft adapted to selectively extend between the flexible tabs whereby the expansion shaft prevents flexion of the flexible tabs such that the laterally extending protrusions secure the surgical implant, and whereby when the expansion shaft does not extend between the flexible tabs, the flexible tabs can be flexed inwardly to cause the laterally extending protrusions to have a narrower profile that is able to be passed through the narrow external opening.

### CROSS-REFERENCE TO RELATED APPLICATIONS

This application claims the benefit of U.S. Provisional Application No. 62/689,707, filed Jun. 25, 2018.

### BACKGROUND OF THE INVENTION

### 1. Field of the Invention

The present invention relates to medical implants, and more particularly to systems and methods for inserting implants such as spinal interbody implants.

### 2. Background and Related Art

One of the difficulties associated with placing certain surgical implants is the difficulty of surgical access. In many instances, surgical access is limited, either by anatomy or by surgical choice to limit tissue damage and to facilitate healing. As a particular example, it can be difficult to place spinal interbody implants, particularly those used for cervical portions of the spine. In such surgeries, access to the interbody space is typically achieved anteriorly, requiring displacement of other body structures. As a result, it often becomes difficult to properly place an interbody implant at the exact desired location. When an implant is not properly placed, the desired surgical result may not be achieved, the patients may experience increased incidence of unwanted side effects, or healing may be delayed.

In some instances, it may also be necessary to apply significant amounts of force to insert a surgical implant such as a cervical interbody implant into a desired location. Surgeons wishing to apply forces to implants during insertion face additional difficulties; it may be difficult to both secure and control the implant while attempting to apply necessary forces to insert the implant. As a result, surgeons may struggle to use existing instruments (often with limited surgical access) to hold, adjust, place, and insert an implant such as a cervical interbody implant. Accordingly, it would be desirable to improve upon existing methods for inserting such implants.

### BRIEF SUMMARY OF THE INVENTION

Implementation of the invention provides surgical implant systems and methods for their use. In particular, implementation of the invention provides surgical implants and implant inserters and methods for using such in surgical implantation procedures. Particular implementations of the invention provide interbody spacer implants and accompanying interbody spacer inserters and methods for using such in spinal fusion procedures. Further particular implementations of the invention provide cervical interbody spacer implants and accompanying interbody spacer inserters and methods for using such in spinal fusion procedures. Implementations of the invention may provide surgical implants alone, implant inserters alone, or systems including both implants and implant inserters.

Implementations of the invention provide an implant inserter adapted to secure and facilitate insertion of a surgical implant having an inserter attachment interface having a narrow external opening at a surface thereof and a broader internal opening. The implant inserter includes a handle and an inserter shaft fixedly attached to and extending from a distal end of the handle. The implant inserter also includes a pair of flexible tabs extending from a distal end of the inserter shaft, the flexible tabs being separated by an inter-tab space, the flexible tabs having laterally extending protrusions adapted to extend into a broader internal opening of a surgical implant and an expansion shaft adapted to selectively extend into the inter-tab space whereby when the expansion shaft extends into the inter-tab space, the expansion shaft prevents flexion of the flexible tabs such that the laterally extending protrusions cannot be pressed together to pass between a narrow external opening of the surgical implant, and whereby when the expansion shaft does not extend into the inter-tab space, the flexible tabs can be flexed into the inter-tab space to cause the laterally extending protrusions to have a narrower profile that is able to be passed through the narrow external opening of the surgical implant.

According to some implementations, a distal end of the expansion shaft slidingly extends into and out of the inter-tab space. According to some implementations, a distal end of the expansion shaft extends into and out of the inter-tab space as a result of an at least partially rotational motion. In some implementations, the surgical implant is a cervical interbody implant.

In some implementations, the inserter shaft includes a channel in the inserter shaft extending from the handle to the distal end of the inserter shaft that receives the expansion shaft. In some implementations, the expansion shaft includes an expansion handle at a proximal end thereof that facilitates manipulation of the expansion shaft within the channel of the inserter shaft. In some implementations, the implant inserter includes a lever operatively attached between the handle and the expansion shaft, whereby manipulation of the lever causes a distal end to selectively extend into the inter-tab space.

Certain implementations of the invention provide an implant inserter adapted to secure and facilitate insertion of a surgical implant having an inserter attachment interface having a narrow external opening at a surface thereof and a broader internal opening. The implant inserter includes a handle and an inserter shaft fixedly attached to and extending from a distal end of the handle. The implant inserter also includes a pair of flexible tabs extending from a distal end of the inserter shaft, the flexible tabs being separated by an inter-tab space, the flexible tabs having laterally extending protrusions adapted to extend into a broader internal opening of a surgical implant and an expansion shaft adapted to selectively contact surfaces of the flexible tabs adjacent the inter-tab space whereby when the expansion shaft contacts surfaces of the flexible tabs adjacent the inter-tab space, the expansion shaft prevents flexion of the flexible tabs such that the laterally extending protrusions cannot be pressed together to pass between a narrow external opening of the surgical implant, and whereby when the expansion shaft does not contact surfaces of the flexible tabs adjacent the inter-tab space, the flexible tabs can be flexed into the inter-tab space to cause the laterally extending protrusions to have a narrower profile that is able to be passed through the narrow external opening of the surgical implant.

In some implementations, a distal end of the expansion shaft slidingly extends into and out of the inter-tab space to cause contact between the distal end of the expansion shaft and the surfaces of the flexible tabs adjacent the inter-tab space. In some implementations, a distal end of the expansion shaft has a broad profile in a first cross-sectional direction and a narrow profile in an orthogonal cross-sectional direction, whereby a rotational motion of the expansion shaft causes selective contact between the surfaces of the flexible tabs adjacent the inter-tab space and the expansion shaft. In some implementations, the surgical implant is a cervical interbody implant.

In some implementations, the inserter shaft includes a channel in the inserter shaft extending from the handle to the distal end of the inserter shaft that receives the expansion shaft. In some implementations, the expansion shaft includes an expansion handle at a proximal end thereof that facilitates manipulation of the expansion shaft within the channel of the inserter shaft. In some implementations, the implant inserter includes a lever operatively attached between the handle and the expansion shaft, whereby manipulation of the lever causes a distal end to selectively extend into the inter-tab space.

Certain implementations of the invention provide a surgical implant insertion system. The surgical implant insertion system includes a cervical interbody implant and an implant inserter. The cervical interbody implant includes an inserter implant interface having a narrow external opening at a surface of the cervical interbody implant and a broader internal opening that is broader than the narrow external opening and that is in communication with the narrow external opening. The implant inserter includes a handle and an inserter shaft fixedly attached to and extending from a distal end of the handle. The implant inserter also includes a pair of flexible tabs extending from a distal end of the inserter shaft, the flexible tabs being separated by an inter-tab space, the flexible tabs having laterally extending protrusions adapted to extend into a broader internal opening of a surgical implant and an expansion shaft adapted to selectively contact surfaces of the flexible tabs adjacent the inter-tab space whereby when the expansion shaft contacts surfaces of the flexible tabs adjacent the inter-tab space, the expansion shaft prevents flexion of the flexible tabs such that the laterally extending protrusions cannot be pressed together to pass between a narrow external opening of the surgical implant, and whereby when the expansion shaft does not contact surfaces of the flexible tabs adjacent the inter-tab space, the flexible tabs can be flexed into the inter-tab space to cause the laterally extending protrusions to have a narrower profile that is able to be passed through the narrow external opening of the surgical implant.

In some embodiments, a distal end of the expansion shaft slidingly extends into and out of the inter-tab space to cause contact between the distal end of the expansion shaft and the surfaces of the flexible tabs adjacent the inter-tab space. In some implementations, a distal end of the expansion shaft includes a broad profile in a first cross-sectional direction and a narrow profile in an orthogonal cross-sectional direction, whereby a rotational motion of the expansion shaft causes selective contact between the surfaces of the flexible tabs adjacent the inter-tab space and the expansion shaft.

In some implementations, the inserter shaft includes a channel in the inserter shaft extending from the handle to the distal end of the inserter shaft that receives the expansion shaft. In some implementations, the expansion shaft includes an expansion handle at a proximal end thereof that facilitates manipulation of the expansion shaft within the channel of the inserter shaft. In some implementations, the implant inserter includes a lever operatively attached between the handle and the expansion shaft, whereby manipulation of the lever causes a distal end to selectively extend into the inter-tab space.

### BRIEF DESCRIPTION OF THE SEVERAL VIEWS OF THE DRAWINGS

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 shows a perspective view of a representative surgical implant;

FIG. 2 shows a cross-sectional view of a portion of a representative surgical implant;

FIG. 3 shows a perspective view of a representative interbody inserter;

FIG. 4 shows a perspective view of a representative interbody inserter;

FIG. 5 shows a cross-sectional view of a representative interbody inserter inserted into a representative interbody implant;

FIG. 6 shows a cross-sectional view of a representative interbody inserter inserted into a representative interbody implant;

FIG. 7 shows a perspective view of an alternative interbody inserter in a disassembled state;

FIG. 8 shows a perspective view of the interbody inserter of FIG. 7 in an assembled state; and

FIG. 9 shows a cross-sectional view of a representative interbody inserter.

### DETAILED DESCRIPTION OF THE INVENTION

A description of embodiments of the present invention will now be given with reference to the Figures. It is expected that the present invention may take many other forms and shapes, hence the following disclosure is intended to be illustrative and not limiting, and the scope of the invention should be determined by reference to the appended claims.

Embodiments of the invention provide surgical implant systems and methods for their use. In particular, embodiments of the invention provide surgical implants and implant inserters and methods for using such in surgical implantation procedures. Particular embodiments of the invention provide interbody spacer implants and accompanying interbody spacer inserters and methods for using such in spinal fusion procedures. Further particular embodiments of the invention provide cervical interbody spacer implants and accompanying interbody spacer inserters and methods for using such in spinal fusion procedures. Embodiments of the invention may provide surgical implants alone, implant inserters alone, or systems including both implants and implant inserters.

Embodiments of the invention provide an implant inserter adapted to secure and facilitate insertion of a surgical implant having an inserter attachment interface having a narrow external opening at a surface thereof and a broader internal opening. The implant inserter includes a handle and an inserter shaft fixedly attached to and extending from a distal end of the handle. The implant inserter also includes a pair of flexible tabs extending from a distal end of the inserter shaft, the flexible tabs being separated by an inter-tab space, the flexible tabs having laterally extending protrusions adapted to extend into a broader internal opening of a surgical implant and an expansion shaft adapted to selectively extend into the inter-tab space whereby when the expansion shaft extends into the inter-tab space, the expansion shaft prevents flexion of the flexible tabs such that the laterally extending protrusions cannot be pressed together to pass between a narrow external opening of the surgical implant, and whereby when the expansion shaft does not extend into the inter-tab space, the flexible tabs can be flexed into the inter-tab space to cause the laterally extending protrusions to have a narrower profile that is able to be passed through the narrow external opening of the surgical implant.

According to some embodiments, a distal end of the expansion shaft slidingly extends into and out of the inter-tab space. According to some embodiments, a distal end of the expansion shaft extends into and out of the inter-tab space as a result of an at least partially rotational motion. In some embodiments, the surgical implant is a cervical interbody implant.

In some embodiments, the inserter shaft includes a channel in the inserter shaft extending from the handle to the distal end of the inserter shaft that receives the expansion shaft. In some embodiments, the expansion shaft includes an expansion handle at a proximal end thereof that facilitates manipulation of the expansion shaft within the channel of the inserter shaft. In some embodiments, the implant inserter includes a lever operatively attached between the handle and the expansion shaft, whereby manipulation of the lever causes a distal end to selectively extend into the inter-tab space.

Certain embodiments of the invention provide an implant inserter adapted to secure and facilitate insertion of a surgical implant having an inserter attachment interface having a narrow external opening at a surface thereof and a broader internal opening. The implant inserter includes a handle and an inserter shaft fixedly attached to and extending from a distal end of the handle. The implant inserter also includes a pair of flexible tabs extending from a distal end of the inserter shaft, the flexible tabs being separated by an inter-tab space, the flexible tabs having laterally extending protrusions adapted to extend into a broader internal opening of a surgical implant and an expansion shaft adapted to selectively contact surfaces of the flexible tabs adjacent the inter-tab space whereby when the expansion shaft contacts surfaces of the flexible tabs adjacent the inter-tab space, the expansion shaft prevents flexion of the flexible tabs such that the laterally extending protrusions cannot be pressed together to pass between a narrow external opening of the surgical implant, and whereby when the expansion shaft does not contact surfaces of the flexible tabs adjacent the inter-tab space, the flexible tabs can be flexed into the inter-tab space to cause the laterally extending protrusions to have a narrower profile that is able to be passed through the narrow external opening of the surgical implant.

In some embodiments, a distal end of the expansion shaft slidingly extends into and out of the inter-tab space to cause contact between the distal end of the expansion shaft and the surfaces of the flexible tabs adjacent the inter-tab space. In some embodiments, a distal end of the expansion shaft has a broad profile in a first cross-sectional direction and a narrow profile in an orthogonal cross-sectional direction, whereby a rotational motion of the expansion shaft causes selective contact between the surfaces of the flexible tabs adjacent the inter-tab space and the expansion shaft. In some embodiments, the surgical implant is a cervical interbody implant.

In some embodiments, the inserter shaft includes a channel in the inserter shaft extending from the handle to the distal end of the inserter shaft that receives the expansion shaft. In some embodiments, the expansion shaft includes an expansion handle at a proximal end thereof that facilitates manipulation of the expansion shaft within the channel of the inserter shaft. In some embodiments, the implant inserter includes a lever operatively attached between the handle and the expansion shaft, whereby manipulation of the lever causes a distal end to selectively extend into the inter-tab space.

Certain embodiments of the invention provide a surgical implant insertion system. The surgical implant insertion system includes a cervical interbody implant and an implant inserter. The cervical interbody implant includes an inserter implant interface having a narrow external opening at a surface of the cervical interbody implant and a broader internal opening that is broader than the narrow external opening and that is in communication with the narrow external opening. The implant inserter includes a handle and an inserter shaft fixedly attached to and extending from a distal end of the handle. The implant inserter also includes a pair of flexible tabs extending from a distal end of the inserter shaft, the flexible tabs being separated by an inter-tab space, the flexible tabs having laterally extending protrusions adapted to extend into a broader internal opening of a surgical implant and an expansion shaft adapted to selectively contact surfaces of the flexible tabs adjacent the inter-tab space whereby when the expansion shaft contacts surfaces of the flexible tabs adjacent the inter-tab space, the expansion shaft prevents flexion of the flexible tabs such that the laterally extending protrusions cannot be pressed together to pass between a narrow external opening of the surgical implant, and whereby when the expansion shaft does not contact surfaces of the flexible tabs adjacent the inter-tab space, the flexible tabs can be flexed into the inter-tab space to cause the laterally extending protrusions to have a narrower profile that is able to be passed through the narrow external opening of the surgical implant.

In some embodiments, a distal end of the expansion shaft slidingly extends into and out of the inter-tab space to cause contact between the distal end of the expansion shaft and the surfaces of the flexible tabs adjacent the inter-tab space. In some embodiments, a distal end of the expansion shaft includes a broad profile in a first cross-sectional direction and a narrow profile in an orthogonal cross-sectional direction, whereby a rotational motion of the expansion shaft causes selective contact between the surfaces of the flexible tabs adjacent the inter-tab space and the expansion shaft.

In some embodiments, the inserter shaft includes a channel in the inserter shaft extending from the handle to the distal end of the inserter shaft that receives the expansion shaft. In some embodiments, the expansion shaft includes an expansion handle at a proximal end thereof that facilitates manipulation of the expansion shaft within the channel of the inserter shaft. In some embodiments, the implant inserter includes a lever operatively attached between the handle and the expansion shaft, whereby manipulation of the lever causes a distal end to selectively extend into the inter-tab space.

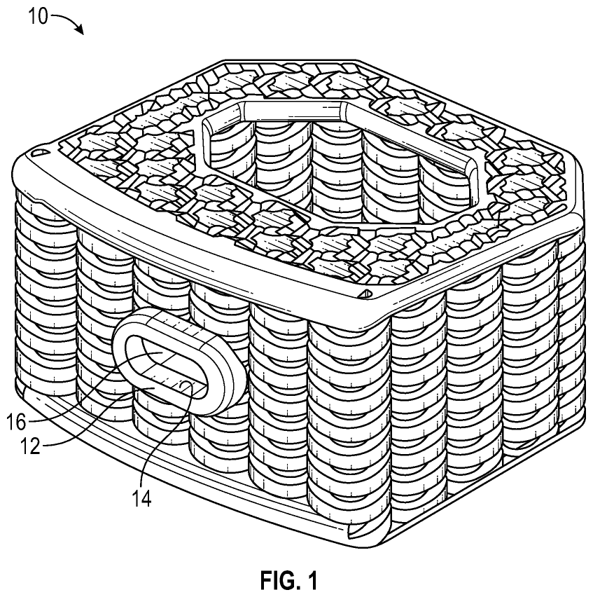

FIG. 1 illustrates one embodiment of a representative implant. In this example, the implant is an interbody spacer 10, in particular a cervical interbody spacer for use in a spinal fusion procedure in the cervical area of the spine. The interbody spacer 10 may be constructed or manufactured in accordance with principles discussed in U.S. patent application Ser. No. 15/372,290, now published as U.S. Patent Application Publication no. US 2017-0156880 A1, which is incorporated herein by reference for all it discloses. In accordance with the principles discussed in that application, the interbody spacer 10 may have a porosity and stiffness that approximates the porosity and stiffness of bone, and leads to improved bone ingrowth and ongrowth to the interbody spacer 10, leading to improved surgical outcomes when used in spinal fusion procedures.

The interbody spacer 10 includes an inserter implant interface 12 on a surface of the interbody spacer 10. In this example, the inserter implant interface 12 is located on an anterior surface of the interbody spacer 10, whereby the interbody spacer 10 may be secured on an interbody insertion instrument during insertion and/or manipulation of the interbody spacer 10 during a surgical procedure. The inserter implant interface 12 includes an external opening 14 and an internal opening 16. The external opening 14 and internal opening 16 are in communication, which is to say that the external opening 14 and the internal opening 16 define a continuous open space into which a portion of the interbody insertion instrument can be inserted, as is discussed in more detail below. Accordingly, the external opening 14 extends from an anterior surface of the interbody spacer 10 posteriorly a certain depth, and the internal opening 16 extends thereafter further posteriorly into the interbody spacer 10.

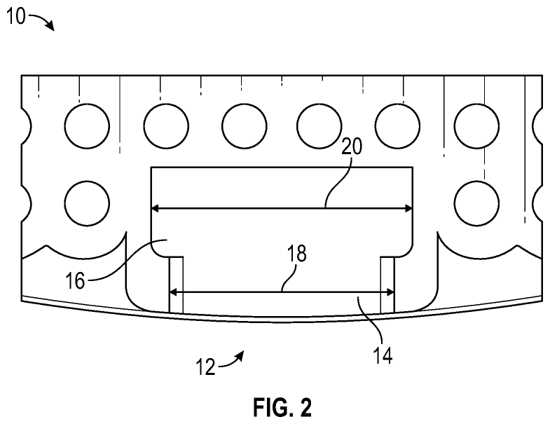

FIG. 2 provides a cross-sectional view, taken generally along a transverse plane (taken with respect to the interbody spacer 10 as it would sit in the human body), of a portion of the interbody spacer 10 of FIG. 1 at the location of the inserter implant interface 12. As may be seen in FIG. 2, the external opening 14 has a width 18 that is narrower than a width 20 of the internal opening 16. In this example, the differences in dimension of the external opening 14 and the internal opening 16 occur generally on the transverse plane of the implant; however, differences in dimensions between the external opening 14 and the internal opening 16 may occur in any direction, including generally on the sagittal plane or in any other desired direction.

A transition in dimensions between the external opening 14 and the internal opening 16 may be abrupt in some embodiments or varying degrees of smooth in different embodiments. The external opening 14 and the internal opening 16 may be formed using any desired process of manufacture, including, without limitation, milling and other subtractive processes. In some embodiments, the external opening 14 and the internal opening 16 are formed by an additive manufacturing process as the interbody spacer 10 is formed by an additive manufacturing process, whereby the external opening 14 and the internal opening 16 are formed as negative space as the interbody spacer 10 is formed by the additive manufacturing process.

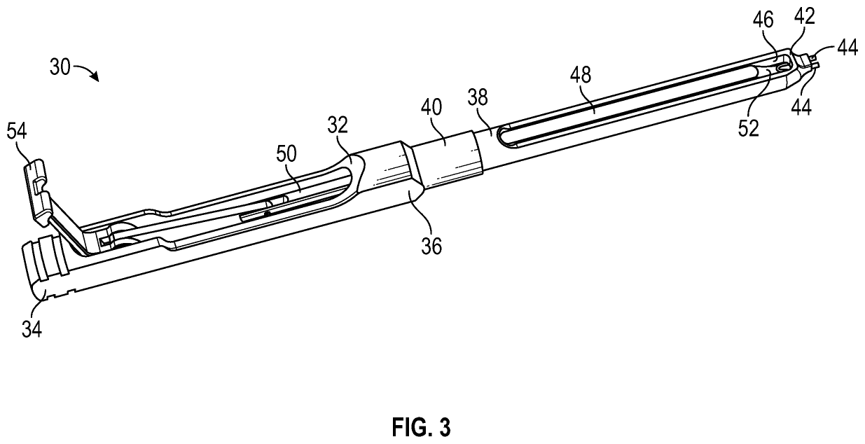

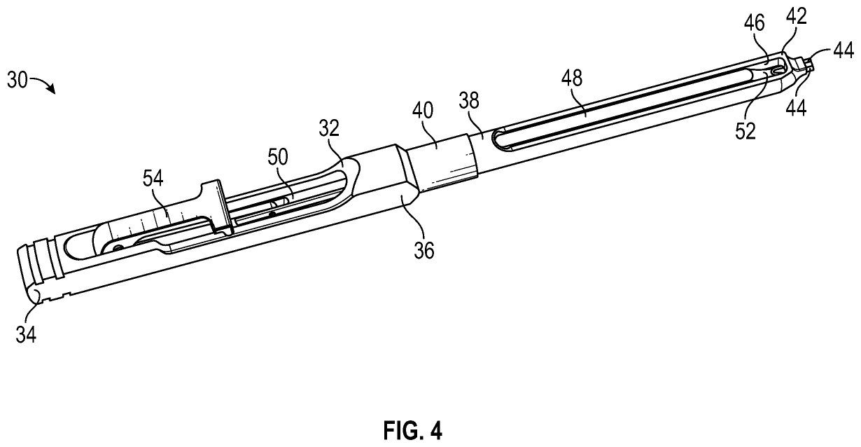

The differences in the dimensions between the external opening 14 and the internal opening 16 allow the interbody spacer 10 to be secured on an insertion instrument. FIGS. 3 and 4 illustrate one embodiment of an insertion instrument, namely an interbody inserter 30. The interbody inserter 30 of various embodiments takes various forms to facilitate manipulation of the interbody inserter 30, to provide desirable ergonomic characteristics, and to achieve desired size relationships for use by the surgeon. Accordingly, the specific embodiment illustrated in FIGS. 3 and 4 is intended to be for purposes of discussion and illustration only, and is not intended to be limiting on the scope of the invention as defined in the appended claims.

The interbody inserter 30 includes a handle 32. The handle 32 is generally adapted to be received in and manipulated by a human hand, and may take any desired shape to permit a surgeon to manipulate the interbody inserter 30. The handle 32 has a proximal end 34 and a distal end 36. An inserter shaft 38 extends generally distally from the distal end 36 of the handle 32. The inserter shaft 38 has a proximal end 40 affixed to the handle 32 and a distal end 42 away from the handle 32. In some embodiments, the inserter shaft 38 is detachable from the handle 32, such as for purposes of sterilization. In other embodiments, the inserter shaft 38 is permanently fixed to the handle 32. In still other embodiments, the inserter shaft 38 is formed with the handle 32.

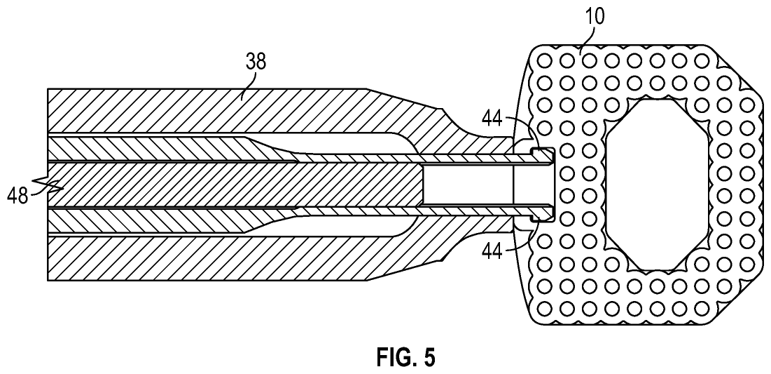

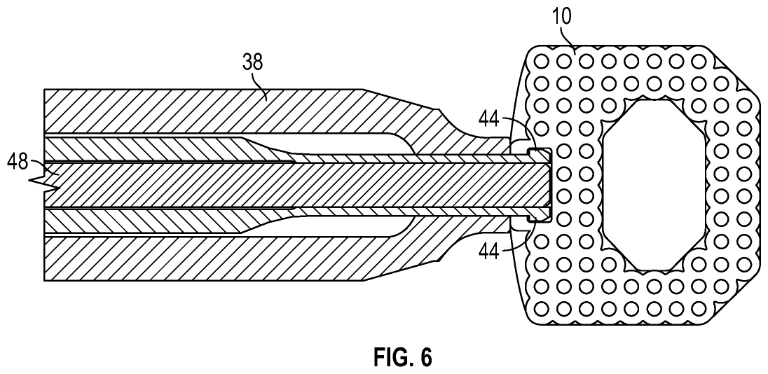

The distal end 42 of the inserter shaft 38 includes a pair of flexible tabs 44. The flexible tabs 44 are co-planar and each include laterally extending protrusions that extend outwardly at the ends of the flexible tabs 44 (as seen more clearly in the cross-sectional views of FIGS. 5 and 6). The flexible tabs 44, and particularly the laterally extending protrusions, are adapted to permit the flexible tabs 44 to be inserted into the external opening 14 of the interbody spacer 10 and are sized and spaced that upon insertion of the flexible tabs 44 into the external opening 14, the flexible tabs 44 are deflected and flexed inward (toward each other) by the inner surface of the external opening 14. As the flexible tabs 44 are inserted fully into the external opening 14, the laterally extending protrusions enter into the internal opening 16, which is wider than the external opening 14, whereby the flexible tabs 44 have space to flex back apart, thereby loosely securing the interbody spacer 10 on the distal end 42 of the inserter shaft 38.

In some embodiments, the inserter shaft 38 is formed of or includes two shaft elements. The first shaft element is an inner shaft that includes the flexible tabs 44. The second shaft element is an outer shaft that slidingly receives the inner shaft therein. The outer shaft of such embodiments has a distal end that is adapted to contact the anterior surface of the interbody spacer 10, whereby when the inner shaft is pulled proximally within the outer shaft, the distal end of the outer shaft contacts the interbody spacer 10 and prevents further proximally oriented motion of the interbody spacer 10, whereby further proximal motion of the inner shaft causes inward deflection of the flexible tabs 44 and removal of the flexible tabs 44 from the interbody spacer 10 and separation of the interbody spacer 10 from the inserter shaft 38.

In other embodiments, the inserter shaft 38 is formed as a single structure. In such embodiments, the inserter shaft 38 is removed from the interbody spacer 10 by application of a withdrawal force to the inserter shaft 38 (e.g., through the handle 32) while the interbody spacer 10 is secured against proximal movement (or, in other words, after surgical placement of the interbody spacer 10, anterior movement). The interbody spacer 10 may be secured against proximal/anterior movement by, for example, a retention force applied to the interbody spacer by the vertebral bodies between which the implanted interbody spacer 10 sits, or by a separate surgical instrument applying a distal force (e.g., a posterior force) to the anterior surface of the interbody spacer 10 in situ.

In general, the force necessary to attach or remove the interbody spacer 10 to or from the inserter shaft 38, deflecting the flexible tabs, is modest but not so high as to cause unwanted motion of the interbody spacer 10 after implantation during a process to remove the interbody inserter 30 from the interbody spacer 10. It is, however, desirable to allow significant forces to be selectively applied to the interbody spacer 10, including anteriorly directed forces as necessary, during an implantation procedure. Accordingly, embodiments of the invention include features to more-firmly secure the interbody spacer 10 on the inserter shaft 38.

In particular, the inserter shaft 38 of interbody inserter 30 illustrated in FIGS. 3 and 4 includes a channel 46 in which sits an expansion shaft 48. The expansion shaft 48 includes a proximal end 50 and a distal end 52. The expansion shaft 48 in this example is adapted to slidingly move distally and proximally within the inserter shaft 38. At a distal-most position of the expansion shaft 48 relative to the inserter shaft 38, the distal end 52 of the expansion shaft 48 extends into an inter-tab space between the two flexible tabs 44, thereby forcing them apart and/or keeping them apart at a distance such that the distance between outer surfaces of the laterally extending protrusions is greater than the width 18 of the external opening 14 (e.g., is approximately equal to or slightly less than the width 20 of the internal opening 16). At a proximal-most position of the expansion shaft 48 relative to the inserter shaft 38, the inter-tab space between the two flexible tabs 44 is vacant, whereby the flexible tabs 44 are permitted to flex toward each other on insertion into or removal from the interbody spacer 10.

In the illustrated embodiment, a lever 54 is operatively attached between the handle 2 and the proximal end 40 of the expansion shaft 48. In this fashion, operation of the lever 54 causes proximal-distal motion of the expansion shaft 48 within the channel 46 and relative to the inserter shaft 38 and flexible tabs 44. FIG. 3 illustrates the lever 54 in the position that causes the expansion shaft 48 to be in its proximal-most position. FIG. 4 illustrates the lever 54 in the position that causes the expansion shaft 48 to be in its distal-most position.

In this way, the interbody spacer 10 can be readily affixed to the interbody inserter 30 by inserting the flexible tabs 44 fully into the inserter implant interface 12 while the expansion shaft 48 is in its proximal-most position (FIG. 3), then the expansion shaft is advanced to its distal-most position by advancing the lever 54 (FIG. 4), thereby securely holding the interbody spacer 10 on the end of the interbody inserter 30. The interbody inserter 30 is then manipulated by the surgeon to insert and properly position the interbody spacer 10 between the vertebral bodies, and as may be appreciated, the surgical access requirements are minimized to the minimum dimensions necessary to permit passage of the interbody spacer 10. Once the interbody spacer 10 is properly positioned, the lever 54 is manipulated back to the position of FIG. 3, and the interbody inserter 30 is removed from the interbody spacer 10.

FIGS. 5 and 6 show cross-sectional views of the distal portion of the interbody inserter 30 inserted into a representative version of the interbody spacer 10, taken generally along the transverse plane. FIG. 5 shows the expansion shaft 48 in its proximal-most position (e.g., in the position in which insertion/withdrawal of the flexible tabs 44 to/from the inserter implant interface 12 of the interbody spacer 10 is facilitated). In contrast, FIG. 6 shows the expansion shaft 48 in its distal-most position (e.g., in the position in which the distal end 52 of the expansion shaft 48 is positioned in the inter-tab space between the flexible tabs 44, thereby preventing insertion/withdrawal of the flexible tabs 44 to/from the inserter implant interface 12 of the interbody spacer 10.

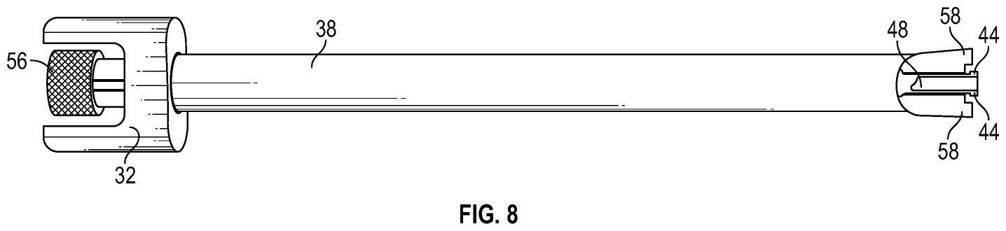

In some embodiments, the engagement of the flexible tabs 44 with the inserter implant interface 12 of the interbody spacer 10 provides sufficient rotational engagement to permit the surgeon to apply any sufficient and desired rotational forces (e.g., around the rotational axis of the expansion shaft 48) to the interbody spacer 10. In other embodiments, it may be desirable to supply additional rotational forces than could be adequately delivered by the flexible tabs 44. Accordingly, in some embodiments, the distal end 42 of the inserter shaft 38 includes one or more distally-extending implant rotation tabs 58 (illustrated in the embodiment of FIG. 8) that are adapted to be received by lateral rotation slots (not show) located on the anterior surface of the interbody spacer 10 on either side of the external opening 14.

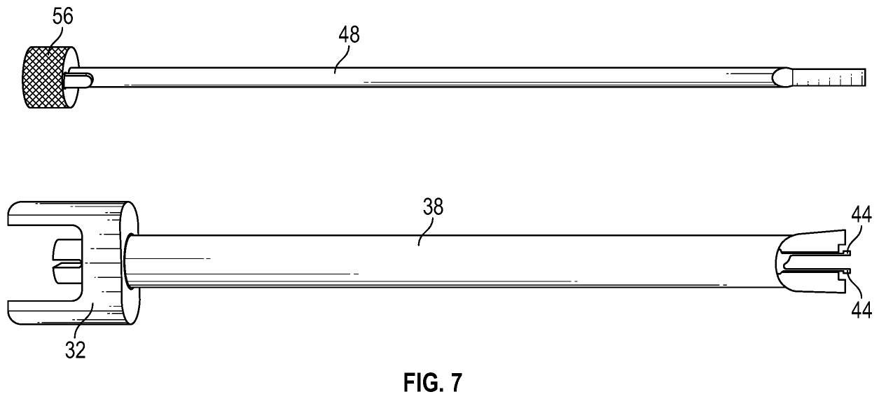

FIGS. 7 and 8 illustrate an alternate embodiment of the interbody inserter 30. In this embodiment, the expansion shaft 48 is removable from the handle 32 and inserter shaft 38, and is operable by a handle 56 of the expansion shaft 48. In this embodiment, the distal end 42 of the expansion shaft 58 has a broad profile in a first cross-sectional direction and a narrow profile in a second, orthogonal cross-sectional direction, such that the expansion shaft 48 can be rotated within the inserter shaft 38 (e.g., by rotation of the handle 56) such that the broad profile or the narrow profile is selectively in-line with the flexible tabs 44. In this embodiment, when the broad profile of the distal end 42 is in-line with the flexible tabs 44, the outer surface of the distal end 42 contacts the surfaces of the flexible tabs 44 adjacent the inter-tab space, thereby forcing the flexible tabs 44 outward or securing them against inward motion. When the narrow profile of the distal end 42 is in-line with the flexible tabs 44, the outer surface of the distal end 42 is spaced apart from the flexible tabs 44 and permits the flexible tabs to flex inward (e.g., permits insertion of the flexible tabs 44 into the external opening 14 or permits withdrawal of the flexible tabs 44 from the external opening 14). Accordingly, securing of the interbody spacer 10 to the interbody inserter 30 for implantation of the interbody spacer 10 and then release of the interbody spacer 10 for removal of the interbody inserter 30 from the interbody spacer can be achieved in this embodiment either by a rotation of the expansion shaft 48 within the inserter shaft 38 or by advancing/withdrawing the expansion shaft 58 within the inserter shaft 38.

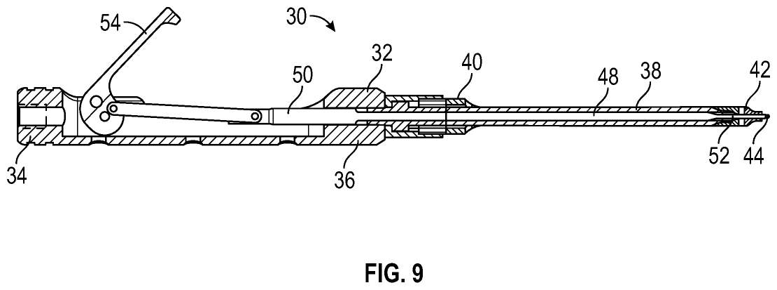

FIG. 9 shows a cross-sectional view of one embodiment of the interbody inserter 30, showing one manner in which the lever 54 may be operatively connected between the handle 32 and the proximal end 50 of the expansion shaft 48. It should be understood that this particular embodiment is intended merely to illustrate one manner of actuating movement between the expansion shaft 48 and the inserter shaft 38, and is not intended to be limiting of the invention as claimed in the appended claims.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. An implant inserter adapted to secure and facilitate insertion of a surgical implant having an inserter attachment interface having a narrow external opening at a surface thereof and a broader internal opening, the implant inserter comprising:
a handle;
an inserter shaft fixedly attached to and extending from a distal end of the handle;
a pair of flexible tabs extending from a distal end of the inserter shaft, the flexible tabs being separated by an inter-tab space, the flexible tabs having laterally extending protrusions adapted to extend into a broader internal opening of a surgical implant; and
an expansion shaft adapted to selectively extend into the inter-tab space whereby when the expansion shaft extends into the inter-tab space, the expansion shaft prevents flexion of the flexible tabs such that the laterally extending protrusions cannot be pressed together to pass between a narrow external opening of the surgical implant, and whereby when the expansion shaft does not extend into the inter-tab space, the flexible tabs can be flexed into the inter-tab space to cause the laterally extending protrusions to have a narrower profile that is able to be passed through the narrow external opening of the surgical implant.

2. The implant inserter as recited in claim 1, wherein a distal end of the expansion shaft slidingly extends into and out of the inter-tab space.

3. The implant inserter as recited in claim 1, wherein a distal end of the expansion shaft extends into and out of the inter-tab space as a result of an at least partially rotational motion.

4. The implant inserter as recited in claim 1, wherein the inserter shaft comprises a channel in the inserter shaft extending from the handle to the distal end of the inserter shaft that receives the expansion shaft.

5. The implant inserter as recited in claim 4, wherein the expansion shaft comprises an expansion handle at a proximal end thereof that facilitates manipulation of the expansion shaft within the channel of the inserter shaft.

6. The implant inserter as recited in claim 4, further comprising a lever operatively attached between the handle and the expansion shaft, whereby manipulation of the lever causes a distal end to selectively extend into the inter-tab space.

7. The implant inserter as recited in claim 1, wherein the surgical implant is a cervical interbody implant.

8. An implant inserter adapted to secure and facilitate insertion of a surgical implant having an inserter attachment interface having a narrow external opening at a surface thereof and a broader internal opening, the implant inserter comprising:
a handle;
an inserter shaft fixedly attached to and extending from a distal end of the handle;
a pair of flexible tabs extending from a distal end of the inserter shaft, the flexible tabs being separated by an inter-tab space, the flexible tabs having laterally extending protrusions adapted to extend into a broader internal opening of a surgical implant; and
an expansion shaft adapted to selectively contact surfaces of the flexible tabs adjacent the inter-tab space whereby when the expansion shaft contacts surfaces of the flexible tabs adjacent the inter-tab space, the expansion shaft prevents flexion of the flexible tabs such that the laterally extending protrusions cannot be pressed together to pass between a narrow external opening of the surgical implant, and whereby when the expansion shaft does not contact surfaces of the flexible tabs adjacent the inter-tab space, the flexible tabs can be flexed into the inter-tab space to cause the laterally extending protrusions to have a narrower profile that is able to be passed through the narrow external opening of the surgical implant.

9. The implant inserter as recited in claim 8, wherein a distal end of the expansion shaft slidingly extends into and out of the inter-tab space to cause contact between the distal end of the expansion shaft and the surfaces of the flexible tabs adjacent the inter-tab space.

10. The implant inserter as recited in claim 8, wherein a distal end of the expansion shaft comprises a broad profile in a first cross-sectional direction and a narrow profile in an orthogonal cross-sectional direction, whereby a rotational motion of the expansion shaft causes selective contact between the surfaces of the flexible tabs adjacent the inter-tab space and the expansion shaft.

11. The implant inserter as recited in claim 8, wherein the inserter shaft comprises a channel in the inserter shaft extending from the handle to the distal end of the inserter shaft that receives the expansion shaft.

12. The implant inserter as recited in claim 11, wherein the expansion shaft comprises an expansion handle at a proximal end thereof that facilitates manipulation of the expansion shaft within the channel of the inserter shaft.

13. The implant inserter as recited in claim 11, further comprising a lever operatively attached between the handle and the expansion shaft, whereby manipulation of the lever causes a distal end to selectively extend into the inter-tab space.

14. The implant inserter as recited in claim 8, wherein the surgical implant is a cervical interbody implant.

15. A surgical implant insertion system comprising:
a cervical interbody implant comprising an inserter implant interface comprising:
a narrow external opening at a surface of the cervical interbody implant; and
a broader internal opening that is broader than the narrow external opening and that is in communication with the narrow external opening; and
an implant inserter comprising:
a handle;
an inserter shaft fixedly attached to and extending from a distal end of the handle;
a pair of flexible tabs extending from a distal end of the inserter shaft, the flexible tabs being separated by an inter-tab space, the flexible tabs having laterally extending protrusions adapted to extend into a broader internal opening of a surgical implant; and
an expansion shaft adapted to selectively contact surfaces of the flexible tabs adjacent the inter-tab space whereby when the expansion shaft contacts surfaces of the flexible tabs adjacent the inter-tab space, the expansion shaft prevents flexion of the flexible tabs such that the laterally extending protrusions cannot be pressed together to pass between a narrow external opening of the surgical implant, and whereby when the expansion shaft does not contact surfaces of the flexible tabs adjacent the inter-tab space, the flexible tabs can be flexed into the inter-tab space to cause the laterally extending protrusions to have a narrower profile that is able to be passed through the narrow external opening of the surgical implant.

16. The surgical implant insertion system as recited in claim 15, wherein a distal end of the expansion shaft slidingly extends into and out of the inter-tab space to cause contact between the distal end of the expansion shaft and the surfaces of the flexible tabs adjacent the inter-tab space.

17. The surgical implant insertion system as recited in claim 15, wherein a distal end of the expansion shaft comprises a broad profile in a first cross-sectional direction and a narrow profile in an orthogonal cross-sectional direction, whereby a rotational motion of the expansion shaft causes selective contact between the surfaces of the flexible tabs adjacent the inter-tab space and the expansion shaft.

18. The surgical implant insertion system as recited in claim 15, wherein the inserter shaft comprises a channel in the inserter shaft extending from the handle to the distal end of the inserter shaft that receives the expansion shaft.

19. The surgical implant insertion system as recited in claim 18, wherein the expansion shaft comprises an expansion handle at a proximal end thereof that facilitates manipulation of the expansion shaft within the channel of the inserter shaft.

20. The surgical implant insertion system as recited in claim 18, further comprising a lever operatively attached between the handle and the expansion shaft, whereby manipulation of the lever causes a distal end to selectively extend into the inter-tab space.
